(19) United States
(12) Patent Application Publication
(10) Pub. No.: US 2006/0047292 A1
(43) Pub. Date: Mar. 2, 2006

(54) Tongue scraper and method of use

(76) Inventor: Devin Reed, South Jordan, UT (US)

Correspondence Address: MORRISS O'BRYANT COMPAGNI, P.C., 136 SOUTH MAIN STREET, SUITE 700, SALT LAKE CITY, UT, 84101, US

(21) Appl. No.: 10/933,078

(22) Filed: Sep. 2, 2004

(51) Int. Cl. A61B 17/24

(52) U.S. Cl. 606/161

(57) ABSTRACT

The present invention includes embodiments of a tongue scraper and methods of using a tongue scraper. Embodiments of a tongue scraper according to the present invention may be included on the top surface of an elongated member or handle of a toothbrush, i.e., on the side opposite the bristles. According to other embodiments of the present invention a tongue scraper may be placed on one or more surfaces of an elongated member having a handle that does not include bristles for cleaning teeth. Embodiments of a tongue scraper may include patterned or random indentations or through-holes in the working surface of the tongue scraper.

### BACKGROUND OF THE INVENTION

1. Field of the Invention

The present invention relates generally to oral hygiene devices. In particular, embodiments of the present invention relate to tongue scrapers and methods of using them.

2. State of the Art

Generally, persons who desire good oral hygiene brush their teeth and the use of dental floss to remove debris from in between the teeth that brushing fails to remove. Mouthwashes and chewing gum are also used to kill germs, suppress the growth of harmful bacteria and to reduce halitosis (bad breath). However, even with regular use of such oral hygiene products, some people still develop cavities (tooth decay), gum ailments, such as gingivitis or gum disease and unpleasant odors in their breath.

The “tongue scraper” is another oral hygiene product known in the art. The use of a tongue scraper tends to be less common than use of a tooth brush, dental floss, mouthwash and gum. However, a tongue scraper can provide a person with another approach to maintaining proper oral hygiene that focuses on the tongue.

The surface of the tongue includes a texture with numerous projections, i.e., papillae, that may collect dead cells, decaying food particles, bacteria and plaque, thus, generally providing a breeding ground for bacteria to grow. Daily tongue cleaning through scraping will remove at least some of the debris from the tongue. Additionally, tongue scraping significantly reduces bacteria and plaque without damaging the tongue.

Conventional tongue scrapers may have one or more raised ridges for dragging across a tongue. These conventional ridges rise up from the top surface of the handle or elongated member of the conventional tongue scraper. Other conventional tongue scrapers may include a working surface formed of raised features including bristles, parallel ridges, spade headed bristles, a scouring pad-like surface, loop pile, flexible posts and the like.

It would be advantageous to have a tongue scraper that could easily be retrofitted to conventional toothbrush designs having indentations or through-holes rather than raised features. Thus, there exists a need in the art for such a tongue scraper and methods of using such a tongue scraper.

### BRIEF SUMMARY OF THE INVENTION

Embodiments of tongue scrapers and methods of using them are disclosed. An embodiment of a tongue scraper may include an elongated member having a proximal end and a distal end and a working surface on the elongated member located near the distal end comprising a plurality of indentations disposed into the working surface.

A method of using a tongue scraper is also disclosed. The method may include providing a tongue scraper, the tongue scraper including an elongated member having a proximal end, a distal end and a working surface on the elongated member located near the distal end comprising a plurality of indentations disposed into the working surface. The method may further include dragging the working surface of the tongue scraper over a surface of a tongue in a direction substantially parallel to a long axis of the elongated member.

### BRIEF DESCRIPTION OF THE SEVERAL VIEWS OF THE DRAWINGS

The following drawings illustrate exemplary embodiments for carrying out the invention. Like reference numerals refer to like parts in different views or embodiments of the present invention in the drawings.

FIG. 1 illustrates a side view of an embodiment of a tongue scraper according to the present invention.

FIG. 2 is an exploded side view of a portion of a working surface illustrating profiles of various embodiments of indentations or through-holes consistent with tongue scrapers of the present invention.

FIG. 3 illustrates perspective views of exemplary shapes of indentations and exemplary arrays of indentations on exemplary working surfaces according to embodiments of tongue scrapers of the present invention.

FIG. 4 illustrates additional top views of shapes suitable for indentations consistent with embodiments of the tongue scraper of the present invention.

FIG. 5 is a flowchart of an embodiment of a method of using a tongue scraper according to the present invention.

FIG. 6 illustrates two perspective photographic images of an embodiment of a tongue scraper according to the present invention where the working surface includes indentations in form of slots formed into the working surface.

### DETAILED DESCRIPTION OF THE INVENTION

The present invention includes embodiments of a tongue scraper and methods of using a tongue scraper. Embodiments of a tongue scraper according to the present invention may be included on the top surface of an elongated member or handle of a toothbrush, i.e., on the side opposite the bristles. According to other embodiments of the present invention a tongue scraper may be placed on one or more surfaces of an elongated member having a handle that does not include bristles for cleaning teeth. Embodiments of a tongue scraper may include patterned or random indentations or through-holes in the working surface of the tongue scraper.

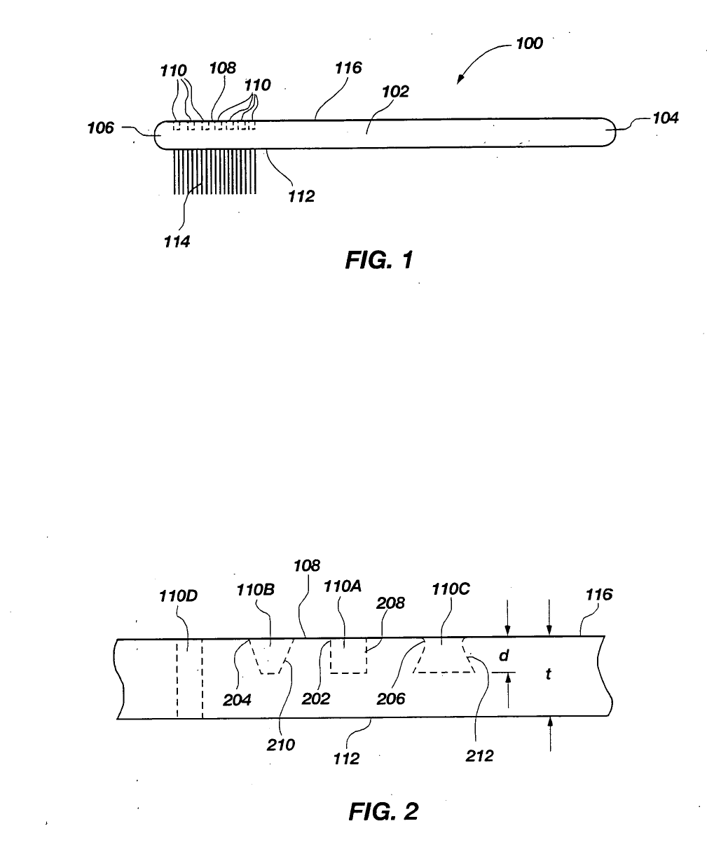

FIG. 1 illustrates a side view of an embodiment of a tongue scraper, shown generally at 100, according to the present invention which may also perform the function of a tooth brush. As shown in FIG. 1, the embodiment of a tongue scraper 100 may include an elongated member 102 having a proximal end 104 and a distal end 106. Tongue scraper 100 may also have a working surface 108 including a plurality of indentations 110 (as shown in FIG. 1, or alternatively through-holes, see FIG. 2 110D and related discussion below) in the distal end 106. The bottom surface 112 of the elongated member 102 may include bristles 114 used for cleaning teeth according to an embodiment of the present invention. Such an embodiment of a tongue scraper 100 may combine the functionality of a conventional toothbrush and the functionality of tongue scrapers 100 as described herein. The proximal end 104 may form a handle for gripping the tongue scraper 100 during use according to an embodiment of the present invention. An alternative embodiment of the tongue scraper (not shown for simplicity) may include all of the above-described elements except for the bristles 112 and, thus, may serve the purpose of tongue scraper without the toothbrush functionality.

Conventional tongue scrapers have ridges, bristles, pads, and other extended structural features extending up from the upper surface of an elongated structural member which may form the handle of such conventional tongue scrapers. A feature common with all of the tongue scrapers 100 according to embodiments of the present invention is that the working surface 108 does not rise above the top surface 112 of the elongated member 102. Rather, indentations 110 or through-holes 110D (see FIG. 2) are formed into the working surface 108 of the tongue scraper 100.

FIG. 2 is an exploded side view of a portion of a working surface 108 illustrating profiles of various embodiments of indentations or through-holes consistent with tongue scrapers 100 of the present invention. The embodiment of an indentation 110A shown in FIG. 2 is consistent with cylindrical, square or other shaped indentations having parallel (or vertical as shown in FIG. 2) sidewalls 208 according to the present invention. Embodiments of the tongue scraper may include corners 202, 204 and 206 having a selective sharpness. The sharpness may be measured by an angle formed at the interface between an indentation 110 and the top surface, for example a medium sharpness may be formed with a corner 202 having a 90° angle. According to other embodiments of tongue scrapers 100 of the present invention, less sharp corners may be formed with obtusely angled (greater than 9020 angle) corners 204. Conversely, sharpness may be increased by employing acutely angled corners 206 according to yet other embodiments of the present invention.

Alternatively, relative sharpness may be measured by selectively radiused or rounded corners (see, corner 206 in FIG. 2, for example) for improved user comfort depending on the material used for the elongated member 102 and as desired. Embodiments of a selectively radiused corner 202, 204, 206 may include a radius of between about 10 μm to about 1000 μm. Selectively radiused corners indentation 110B as shown in FIG. 2 is consistent with a truncated cone, truncated pyramid and other shapes having obtusely angled corners 204 and open-faced sidewalls 210 according to embodiments of the present invention. Again, the obtusely angled corners 204 may be relatively sharp or rounded as desired according to additional embodiments of the present invention. Indentation 110C is consistent with any preselected shape of indentation having acutely angled corners 206 and closed-faced sidewalls 210. Generally, a rounded acutely angled corner 206 will be preferable to a relatively sharp angle for user comfort associated with tongue scrapers 100 including indentations having the characteristics of indentation 110C.

Also shown in FIG. 2 is an embodiment of a through-hole 110D which may be incorporated in a working surface 108 on a tongue scraper 100 according to the present invention. The through-hole 110D embodiment of an indentation 110 allows for easier cleaning of the tongue scraper 100. Each through-hole 110D may have a cylindrical, square-tube or rectangular tube shaped profile according to embodiments of the present invention. Through-hole 110D may have a funnel shaped profile, that is, a larger opening at the top surface 116 and a smaller opening at the bottom surface 112 according to another embodiment of a tongue scraper 100 of the present invention. Such a funnel shaped profile may be useful for supporting bristles 114 by maximizing closed surface area in the bottom surface 112 of an elongated member 102 according to various embodiments of the present invention.

Another embodiment of a tongue scraper 100 according to the present invention is disclosed. The embodiment of a tongue scraper 100 may include an elongated member 102 having a proximal end 104 and a distal end 106 and a working surface 108 on the elongated member 102 located near the distal end 106 including a plurality of indentations 110 disposed into the working surface 108. According to another aspect of the present invention, the working surface 108 may be located on more than one side of the elongated member. For example and not by way of limitation, an embodiment of a tongue scraper 100 according to the present invention may include a working surface 108 on the top surface 116 and on both sides of the distal end 106 that are adjacent the working surface 108.

Indentations 110 may take the form of through-holes 110D passing through the distal end 106 of the elongated member 102 from the working surface 108 or top surface 116 all the way to the bottom surface 112. Profiles of the through-holes 110D may be cylindrically shaped, square tube shaped, conically shaped, pyramid shaped, Aztec pyramid or stepped shaped, inverted or otherwise according to various embodiments of the present invention. Other embodiments of the present invention may include through-holes 110D in the form of parallel slots, wherein each of the parallel slots may form an opening in the working surface 108, wherein the openings comprise a shape when viewed from above the working surface. Those shapes may include an oval, wavy line or a zig-zag line. Of course one skilled in the art will recognize that many other variations on the profiles and shapes of openings of through-holes consistent with embodiments of the present invention are also possible and are considered to be within the scope of the present invention.

Referring again to FIG. 2, embodiments of tongue scrapers 100 according to the present invention may include a plurality of indentations 110 wherein the indentations 110 have parallel sidewalls, for example indentations 110A and 110D. Other embodiments of tongue scrapers 100 according to the present invention may include a plurality of indentations 110 wherein the indentations 110 have obtusely angled corners and open-faced sidewalls such as indentation 110B. Still other embodiments of tongue scrapers 100 according to the present invention may include a plurality of indentations 110 wherein the indentations 110 have acutely angled corners and closed-faced sidewalls, for example indentation 110C. Yet other embodiments of tongue scrapers 100 according to the present invention may include a plurality of indentations 110 having corners that are relatively sharp 202, 204 or alternatively rounded or radiused 206 to increase user comfort. The depth, d, of the indentations may be from about a fraction of a millimeter to the thickness, t, of the elongated member 102, according to embodiments of the present invention. For example, in the embodiment of a tongue scraper 100 having through-hole indentations 110D, the depth, d, of a through-hole 110D is equal to the thickness of the elongated member 102, i.e., d=t.

According to additional embodiments of the tongue scraper 100 of the present invention each of the plurality of indentations may comprise a shape when viewed from above the working surface 108. For example and not by way of limitation, the shape may take the form of a star, a square, a circle, a triangle, a heart, a rectangle, an oval, a Christmas tree, an octagon, a peanut, a house, an animal, a dinosaur, a wave, a dog bone, a flower, a clover, an hour glass, a letter, a number or a symbol. Such shapes may be in the form of a two-dimensional uniform array on the working surface 108 according to embodiments of the present invention. Such shapes may be randomly patterned on the working surface 108 according to other embodiments of the present invention. Still other embodiments may include randomly mixed shapes in uniform two-dimensional arrays or in random patterns according to the present invention.

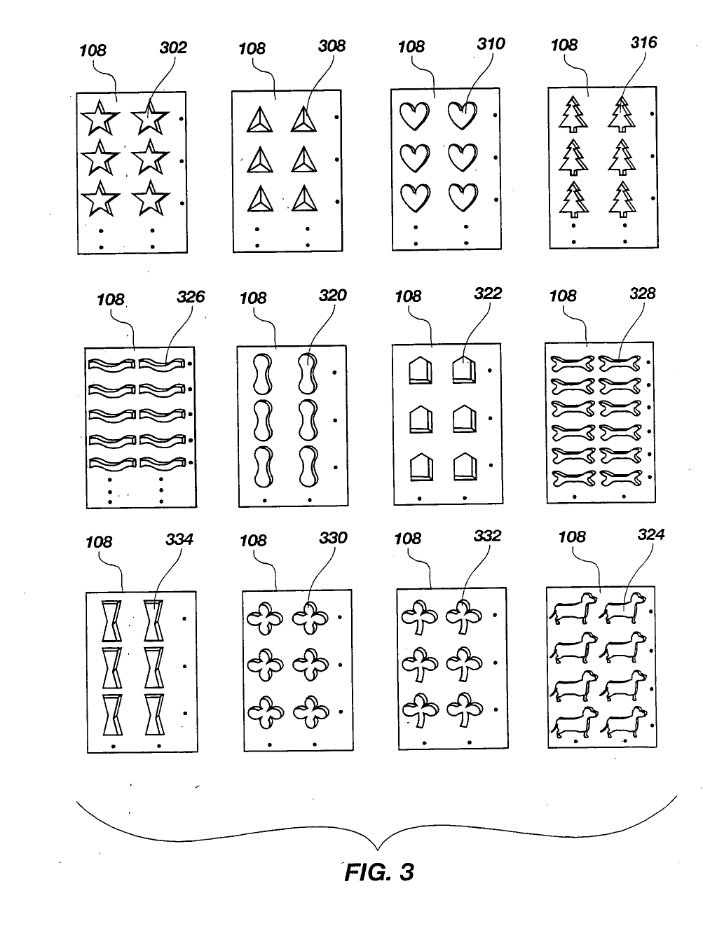

FIG. 3 illustrates perspective views of exemplary shapes of indentations 110 and exemplary arrays of indentations 110 on exemplary working surfaces 108 according to embodiments of tongue scrapers 100 of the present invention. For example, indentations 110 may take the shape of a rounded internal point star 302 and could be arrayed on a working surface as shown in FIG. 3 according to an embodiment of a tongue scraper according to the present invention. In other embodiments of a tongue scraper 100 according to the present invention, indentations 110 may take the shape of triangles with open faced sidewalls 308, hearts with rounded internal points 310, Christmas trees with rounded internal points 316, peanuts 320, houses 322, animals 324, waves 326, dog bones 328, flowers 330, clovers 332 or hour glasses 334.

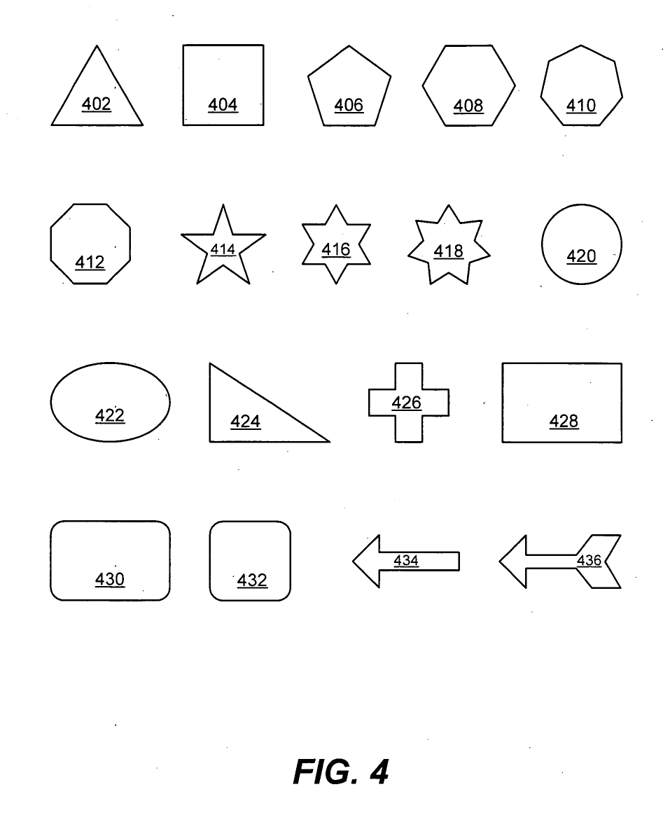

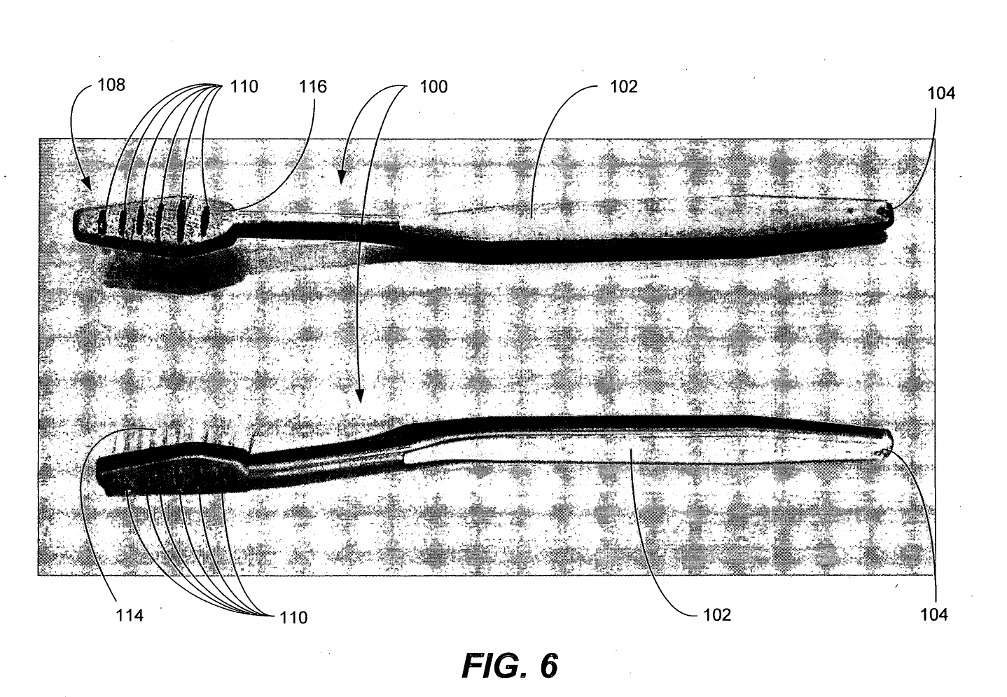

FIG. 4 illustrates additional top views of shapes suitable for indentations 110 consistent with embodiments of tongue scrapers 100 according to the present invention. For example, indentations 110 may be formed in the shape of a triangle 402, square 404, pentagon 406, hexagon 408, heptagon 410, octagon 412, 5-point star 414, 6-point star 416, 7-point star 418, circle 420, oval 422, right-sided triangle 424, plus sign 416, rectangle 428, rounded corner rectangle 430, rounded corner square 432, arrow 434, arrow with tail 436, or slot (see FIG. 6 and related discussion below).

Those shapes shown in FIGS. 3 and 4 are merely exemplary and there are many other possible shapes that could be used consistent with the present invention. One skilled in the art will recognize that any suitable shape, symbol, character or marking may be used consistent with the embodiments of the present invention. All such variations are considered to be within the scope of the present invention. Additionally, the shape selected for particular indentations 110 of a given embodiment of a tongue scraper 100 may be chosen to coincide with a decorative or seasonal theme associated with the intended sale of the tongue scraper 100. For example, the use of Christmas tree 316 shaped indentations 110 may coincide with an embodiment of a Christmas themed tongue scraper 100. Similarly, a heart 310 shaped indentation 110 may be used to coincide with a Valentines Day embodiment of a tongue scraper 100 and a star 302 shaped indentation 110 may be used to coincide with a 4^{th} of July Holiday embodiment of a tongue scraper consistent with the present invention.

Yet another embodiment of the present invention is a tongue scraper 100 including bristles 114 for brushing teeth located on a bottom surface 112 of the elongated member 102. The bristles 114 may be formed of any conventional bristle materials as known to one of ordinary skill in the art. The number and location of bristles 114 are not critical to the embodiments of the present invention and may be selected in any manner known to one of skill in the art.

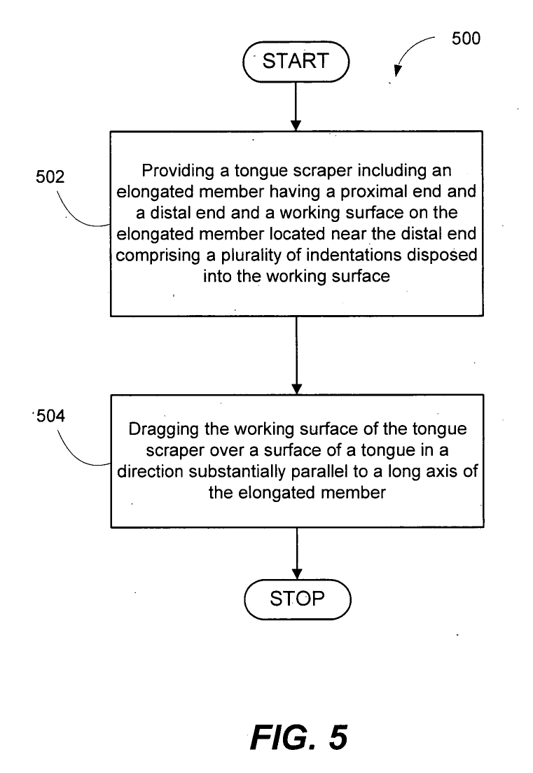

FIG. 5 is a flowchart of an embodiment of a method 500 of using a tongue scraper according to the present invention. Method 500 may include providing 502 a tongue scraper 100, the tongue scraper 100 including an elongated member 102 having a proximal end 104 and a distal end 106 and a working surface 108 on the elongated member 102 located near the distal end 106 comprising a plurality of indentations 110 disposed into the working surface 108. Method 500 may further include dragging 504 the working surface 108 of the tongue scraper 100 over a surface of a tongue in a direction substantially parallel to a long axis of the elongated member 102.

Method 500 may further include providing a tooth paste on the working surface 108 prior to dragging 504 according to another embodiment of method 500. According to yet another embodiment method 500 may further include dragging the working surface 108 back and forth across the surface of the tongue. Yet other embodiments of method 500 may include dragging the working surface 108 of the tongue scraper 100 over a surface inside a cheek and opposite tooth gums according to the present invention. Thus, the tongue scraper 100 of the present invention may be used to clean other soft tissue in the user's mouth. For example in yet another embodiment, method 500 may further include dragging the working surface 108 of the tongue scraper 100 over a surface of tooth gums according to the present invention.

FIG. 6, illustrates two perspective photographic images of an embodiment of a tongue scraper 100 where the working surface 108 includes indentations 110 in form of slots formed into the working surface 108. The working surface 108 may be on the top surface 116 of a toothbrush having bristles 114 as shown in FIG. 6. Furthermore, as can be seen from FIG. 6, the tongue scraper 100 of the present invention can easily be retrofitted to existing or conventional toothbrushes.

An advantage of the tongue scrapers 100 described herein is that the tongue scraper functionality, i.e., a working surface 108 with indentations 110 or through-holes 110D formed into the elongated member 102, may be retrofitted to existing conventional toothbrush designs with little effort. As known to those of ordinary skill in the art, the indentations 110 or through-holes 110D may be stamped, punched, cut or drilled into the top surface 116 of a plastic or plastic like material of an elongated member 102 using stamps, dies, drills or other micromachining tools. Alternatively, the indentations 110 or through-holes 110D may be manifested in the molds used to form a tongue scraper 100 during an injection molding process. Of course, those skilled in the art will readily recognize alternative methods of fabricating tongue scrapers 100 having indentations 110 or through-holes 110D in a working surface 108 of an elongated member 102 as described herein.

While the foregoing advantages of the present invention are manifested in the illustrated embodiments of the invention, a variety of changes can be made to the configuration, design and construction of the invention to achieve those advantages. Hence, reference herein to specific details of the structure and function of the present invention is by way of example only and not by way of limitation.

What is claimed is:

1. A tongue scraper, comprising:
an elongated member having a proximal end and a distal end; and
a working surface on the elongated member located near the distal end comprising a plurality of indentations disposed into the working surface, wherein corners formed by the plurality of indentations in the working surface have a selected sharpness.

2. The tongue scraper according to claim 1, wherein the plurality of indentations comprise through-holes.

3. The tongue scraper according to claim 2, wherein the through-holes comprise conically shaped profiles.

4. The tongue scraper according to claim 2, wherein the through-holes comprise pyramid shaped profiles.

5. The tongue scraper according to claim 2, wherein the through-holes comprise inverted Aztec pyramid or stepped shaped profiles.

6. The tongue scraper according to claim 2, wherein the through-holes comprise cylindrically shaped profiles.

7. The tongue scraper according to claim 2, wherein the through-holes comprise square tube profiles.

8. The tongue scraper according to claim 2, wherein the through-holes comprise parallel slots, wherein each of the parallel slots form an opening in the working surface, wherein the openings comprise a shape when viewed from above the working surface, wherein the shape comprises at least one of a wavy line or a zig-zag line.

9. The tongue scraper according to claim 1, wherein the plurality of indentations comprise parallel sidewalls.

10. The tongue scraper according to claim 1, wherein the plurality of indentations each comprise profiles having obtusely angled corners and open-faced sidewalls.

11. The tongue scraper according to claim 10, wherein the corners are rounded or radiused to a radius from between about 10 μm to about 1000 μm.

12. The tongue scraper according to claim 1, wherein the plurality of indentations each comprise profiles having acutely angled corners and closed-faced sidewalls.

13. The tongue scraper according to claim 12, wherein the corners are rounded or radiused.

14. The tongue scraper according to claim 1, wherein each of the plurality of indentations comprise a shape when viewed from above the working surface, wherein the shape comprises at least one of a star, a square, a circle, a triangle, a heart, a rectangle, an oval, a Christmas tree, an octagon, a peanut, a house, an animal, a dinosaur, a wave, a dog bone, a flower, a clover, an hour glass, a letter, a number or a symbol.

15. The tongue scraper according to claim 1, further comprising bristles for brushing teeth on a bottom surface of the elongated member.

16. A method of using a tongue scraper, comprising:
providing a tongue scraper, the tongue scraper comprising:
an elongated member having a proximal end and a distal end; and
a working surface on the elongated member located near the distal end comprising a plurality of indentations disposed into the working surface;
dragging the working surface of the tongue scraper over a surface of a tongue in a direction substantially parallel to a long axis of the elongated member.

17. The method according to claim 16, further comprising providing a tooth paste on the working surface prior to dragging.

18. The method according to claim 16, further comprising dragging the working surface back and forth across the surface of the tongue.

19. The method according to claim 16, further comprising dragging the working surface of the tongue scraper over a surface inside a cheek and opposite tooth gums.

20. The method according to claim 16, further comprising dragging the working surface of the tongue scraper over a surface of tooth gums.
